(19) United States
(12) Patent Application Publication
(10) Pub. No.: US 2023/0101497 A1
(43) Pub. Date: Mar. 30, 2023

(54) CARDIAC PACING DEVICE

(71) Applicant: MAXWELL BIOMEDICAL, INC., San Diego, CA (US)

(72) Inventors: Randell L. WERNETH, San Diego, CA (US); Graydon Ernest BEATTY, Bloomington, MN (US); R. Maxwell Flaherty, Topsfield, MA (US); J. Christopher Flaherty, Nottingham, NH (US)

(21) Appl. No.: 17/908,002

(22) PCT Filed: Mar. 9, 2021
(86) PCT No.: PCT/US2021/021467

§ 371 Date: Aug. 30, 2022

Related U.S. Application Data
(60) Provisional application No. 62/987,238, filed on Mar. 9, 2020.

(51) Int. Cl. A61N 1/368; A61N 1/05; A61N 1/378; A61N 1/37

(52) U.S. Cl. CPC A61N 1/368; A61N 1/057; A61N 1/3787; A61N 1/3702

(57) ABSTRACT

Provided herein are systems for providing therapy to the heart of a patient. The systems include an implantable device for implantation proximate the heart of the patient. The implantable device includes: an anchoring element for maintaining the position of the implantable device after implantation in the patient, at least one sensing electrode for sensing the electrical activity of the heart, at least three pacing electrodes for delivering electrical energy to the tissue of the heart, and a controller including an algorithm for determining when the patient requires therapy. The systems further include an external device having a transceiver for transmitting energy to the implantable device.

### RELATED APPLICATIONS

The present application claims priority to U.S. Provisional Patent Application Ser. No. 62/987,238, entitled “Stent, Mounted and Delivered Wireless, Batteryless Micropacing Chip System”, filed Mar. 9, 2020, the content of which is incorporated herein by reference in its entirety for all purposes.

The present application, while not claiming priority to, may be related to U.S. Provisional Patent Application Ser. No. 63/032,687, entitled “Rechargeable Biomedical Battery Powered Wireless Self-Anchoring Micro-pacing and Sensing Devices and Control System”, filed May 31, 2020, the content of which is incorporated herein by reference in its entirety for all purposes.

The present application, while not claiming priority to, may be related to International PCT Patent Application Serial Number PCT/US2020/049349, entitled “Cardiac Stimulation System”, filed Sep. 4, 2020, which claims priority to U.S.

Provisional Application Ser. No. 62/895,655, filed Sep. 4, 2019, each of which is hereby incorporated by reference.

### FIELD OF THE INVENTION

The present inventive concepts relate generally to medical systems, and in particular systems and devices used to pace or otherwise treat the heart of a patient.

### BACKGROUND

The heart is a critical muscle in humans and many other animals that is responsible for circulating blood through the circulatory system. The human heart is made up of four chambers, two upper atria, and two lower ventricles, organized into a left and right pairing of an atrium and a ventricle. In a healthy heart, the chambers contract and relax in a synchronized fashion, referred to as a “beat,” in order to force blood through the network of veins and arteries.

Irregular heartbeats can pose a health risk, and in some cases regular beating can be restored via electrical stimulation. Implantable devices called “pacemakers” are devices which can stimulate the muscle tissue, causing it to contract. By carefully and regularly applying stimulation as needed, normal heart rhythm can be restored.

There is a need for improved systems for treating irregular heartbeats.

### SUMMARY

According to an aspect of the present inventive concepts, a system for providing therapy to a heart of a patient, the system comprises: an implantable device configured to be implanted proximate the heart of the patient, the implantable device comprising: an anchoring element configured to maintain the position of the implantable device after implantation in the patient; at least one sensing electrode configured to sense the electrical activity of the heart; at least three pacing electrodes configured to deliver electrical energy to the tissue of the heart; and a controller including an algorithm. The algorithm is configured to determine when the patient requires therapy. The system further comprises an external device comprising a transceiver configured to transmit energy to the implantable device. The implantable device does not comprise a battery.

In some embodiments, the implantable device comprises at least a portion that is configured to be implanted in a blood vessel of the patient, and the at least a portion comprises at least one electrode of the at least three pacing electrodes. The at least a portion can be configured to be implanted in a vein of the patient. The vein can comprise the Vein of Marshall. The at least a portion can comprise a geometry configured to occlude the blood vessel. The at least a portion can comprise a geometry that allows flow of blood through the at least a portion.

In some embodiments, the implantable device comprises at least a first portion that is configured to be implanted in a blood vessel of the patient and at least a second portion that is configured to be implanted on the epicardial surface of the patient's heart, and the first portion comprises at least one electrode of the at least three pacing electrodes, and the second portion comprises at least one electrode of the at least three pacing electrodes.

In some embodiments, the implantable device comprises at least a first portion that is configured to be implanted in a blood vessel of the patient and at least a second portion that is configured to be implanted at a second anatomical location, and the first portion comprises at least one electrode of the at least three pacing electrodes, and the second portion comprises the controller.

In some embodiments, the anchoring element comprises a stent-like structure. The anchoring element can comprise a diameter that tapers between a first portion of the anchoring element and a second portion of the anchoring element.

In some embodiments, the algorithm comprises an artificial intelligence algorithm.

In some embodiments, the external device transceiver is configured to transmit electromagnetic energy to the implantable device.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of a system for diagnosing and/or treating a patient, consistent with the present inventive concepts.

FIG. 2A illustrates a side view of an implantable device including two anchoring assemblies, consistent with the present inventive concepts.

FIG. 2B illustrates a side view of an implantable device including a stent-like anchoring assembly, consistent with the present inventive concepts.

FIG. 2C illustrates a side view of the implantable device of FIG. 2B in a radially collapsed configuration, consistent with the present inventive concepts.

FIG. 3A illustrates a sectional view of an implantable device, consistent with the present inventive concepts.

FIG. 3B illustrates a sectional view of an implantable device, consistent with the present inventive concepts.

FIGS. 4A and 4B illustrate side and sectional views, respectively, of an implantable device comprising a basket-like anchoring element, consistent with the present inventive concepts.

FIGS. 5A and 5B illustrate side views of two implantable devices, each including a single anchoring element, consistent with the present inventive concepts.

FIGS. 6A and 6B illustrate anatomical side views of a partially deployed and a fully deployed implantable device, consistent with the present inventive concepts.

FIG. 7 illustrates a perspective view of an implantable device being deployed from the distal portion of a clinician device, consistent with the present inventive concepts.

FIGS. 8A, 8B, and 8C illustrate side views of various embodiments of stent-like anchoring elements for an implantable device, consistent with the present inventive concepts.

FIG. 9 illustrates a perspective view of an anchoring element for an implantable device, consistent with the present inventive concepts.

FIG. 10 illustrates a perspective view of an implantable device, consistent with the present inventive concepts.

FIGS. 11A, 11B, and 11C illustrate perspective views of three non-circumferential anchoring elements, consistent with the present inventive concepts.

FIG. 12A illustrates a side view of an implantable device, consistent with the present inventive concepts.

FIG. 12B illustrates a top view of a circuit board of an implantable device, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

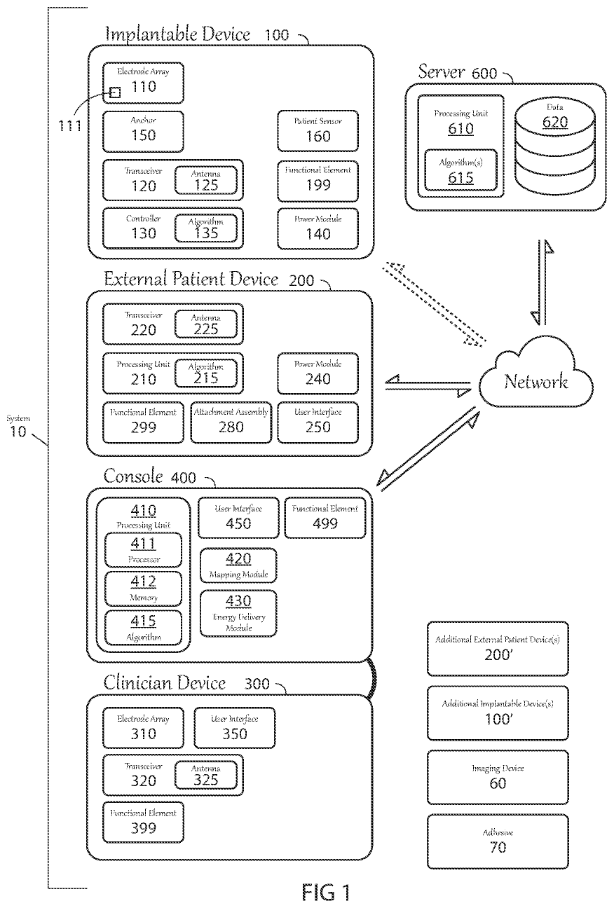

Referring now to FIG. 1, a schematic view of a system for diagnosing and/or treating a patient is illustrated, consistent with the present inventive concepts. System 10 can comprise one or more devices (e.g. devices for a clinician to perform a procedure, devices for a patient to position proximate their body, and/or devices for implantation in the patient) which can be configured to monitor one or more patient parameters, diagnose one or more patient conditions, and/or to treat one or more patient conditions, such as to treat a condition based on one or more patient diagnoses determined by system 10. For example, system 10 can be configured to diagnose and/or treat (“treat” herein) atrial fibrillation (AF), such as by monitoring the electrical activity of the patient's heart, and by pacing the heart muscle to restore sinus rhythm when fibrillation is detected. System 10 can include one or more devices configured to be implanted, implantable device 100, which can be implanted into the patient for an extended period of time (e.g. at least 1 month, at least 3 months, and/or at least 6 months), such as when implanted by a clinician during a clinical procedure. In some embodiments, system 10 comprises one or more externally-placed devices, external patient device 200, which can comprise one or more devices that are configured to diagnose and/or treat a patient from one or more locations outside the patient's body. Alternatively or additionally, external patient device 200 (also referred to as EPD 200) can be configured to communicate (e.g. wirelessly communicate) with implantable device 100 (also referred to as ID 100), such as to transfer data between EPD 200 and ID 100, and/or to transfer power from EPD 200 to ID 100.

System 10 can be configured to detect irregular or otherwise undesirable (“irregular” or “undesirable” herein) electrical conduction patterns in tissue and/or to deliver energy to the tissue to restore a regular (e.g. healthy) electrical conduction pattern. For example, system 10 can be configured to monitor the electrical activity of the heart (e.g. conduction patterns proximate the left atrium of the heart), and to detect the presence of irregular conduction patters, such as conduction patterns indicative of AF. Additionally or alternatively, system 10 can deliver electrical energy (e.g. pacing pulses) to tissue to alter the irregular conduction patterns within the tissue. In some embodiments, system 10 is configured to deliver “multi-site” pacing, where pacing energy is delivered from two or more electrodes positioned at different locations, such as different locations proximate the left atrium. For example, system 10 can be configured to deliver multi-site left-atrial pacing (i.e. delivery of energy to two left atrial tissue locations) configured to restore sinus rhythm in patients experiencing AF. In some embodiments, system 10 comprises one or more devices (e.g. devices 100 and/or 300 described herein) that are configured to ablate tissue, such as by delivering energy configured to thermally ablate and/or irreversibly electroporate tissue (e.g. tissue associated with atrial fibrillation or other arrhythmia of the patient). In these embodiments, system 10 can be further configured to also deliver pacing energy to tissue, such as multi-site pacing energy and/or other pacing energy, such as is described herein.

System 10 can include one or more devices for use by a clinician during a clinical procedure, clinician device 300. Clinician device 300 (also referred to as CD 300) can comprise one or more delivery devices, such as a kit of devices configured to enable the clinician to perform an implantation procedure for implanting ID 100 into the patient. For example, CD 300 can comprise one or more delivery catheters, such as when ID 100 is configured to be implanted during a minimally invasive procedure, such as an interventional procedure performed in a catheterization laboratory (often referred to as a “cath lab”). For example, CD 300 can comprise one or more tools for percutaneous delivery of ID 100 in the patient's vasculature. Alternatively or additionally, CD 300 can comprise one or more surgical tools (e.g. minimally invasive tools) for surgically implanting ID 100 (e.g. in an operating room). In some embodiments, ID 100 comprises a first geometry where ID 100 is in an undeployed state, such as a geometry comprising a collapsed or otherwise undeployed geometry configured to allow ease of insertion into the patient. ID 100 can be configured to transition from the first geometry into a second geometry in which ID 100 is in an expanded or otherwise deployed state.

System 10 can include console 400. Console 400 can operably connect to CD 300 and can be configured to facilitate one or more processes, energy deliveries, data collections, data analyses, data transfers, signal processing, and/or other functions (“functions” herein) of system 10. In some embodiments, system 10 is constructed and arranged to map electrical activity within the body (e.g. electrical activity of the heart), such as when CD 300 comprises a mapping catheter and console 400 comprises mapping module 420. Mapping module 420 can be configured to record and process mapping signals recorded by CD 300. In some embodiments, system 10 is constructed and arranged to ablate tissue (e.g. ablate cardiac tissue to treat AF). In these embodiments, console 400 comprises energy delivery module 430. Energy delivery module 430 can be configured to deliver ablative energy to tissue, such as via one or more energy delivery elements (e.g. electrodes, ultrasound transducers, light-emitting elements, and the like) of CD 300. Console 400 can include processing unit 410, which can be configured to perform one or more functions of console 400 (e.g. as described hereabove). Processing unit 410 can include processor 411, memory 412, and/or algorithm 415, each as shown. In some embodiments, console 400 includes one or more user interfaces, user interface 450. In some embodiments, console 400 includes one or more functional elements, functional element 499 shown. Functional element 499 can include one or more sensors and/or transducers. Functional element 499 can comprise a pumping mechanism, such as a mechanism configured to deliver a pharmaceutical drug, a cooling or warming fluid, an insufflation fluid, and/or other flowable material (e.g. to clinician device 300).

System 10 can include one or more imaging devices, imaging device 60. Imaging device 60 can comprise an imaging device selected from the group consisting of: an X-ray device such as a fluoroscopy device; a CT scanner device; an MRI device; an ultrasound imaging device; and combinations of these.

ID 100 can comprise one or more arrays of functional elements (e.g. sensors and/or transducers), electrode array 110, comprising one, two or more elements, electrodes 111. In some embodiments, one or more components of ID 100 (e.g. the components on the outer surfaces of ID 100 which will be exposed to the environment within the body when implanted) comprise biocompatible materials.

ID 100 can comprise one, two, or more anchoring elements, anchor 150, that is configured to attach to and maintain the position of one or more components of ID 100 (e.g. maintain the position within a vessel). Anchor 150 can include one, two, or more hooks or barbs, adhesive (e.g. fibrin glue), or other anchoring elements. For example, anchor 150 can comprise one two or more scaffolding elements, such as scaffolding elements each comprising a stent-like structure, constructed and arranged to be positioned within a cardiac vessel and to maintain the position of ID 100 within the vessel. Each anchor 150 can comprise a structure (e.g. a stent-like structure) that is plastically deformable (e.g. configured to be expanded by an angioplasty balloon) and/or self-expanding (e.g. comprising self-expanding nickel titanium alloy or other self-expanding material). In some embodiments, ID 100 is constructed and arranged to be implanted into a vein and/or an artery, such as when implanted in the Vein of Marshall. Anchor 150 can be constructed and arranged to maintain the position of ID 100 (e.g. at least a portion of ID 100) within a target blood vessel (e.g. the Vein of Marshall) or other blood vessel (e.g. vein or artery) location. In some embodiments, one or more portions of ID 100 (e.g. a portion including one or more electrodes 111) is configured to be positioned in a blood vessel selected from the group consisting of: a coronary vein; a coronary artery; the Vein of Marshall; the coronary sinus; the great cardiac vein; the anterior intraventricular vein; the middle cardiac vein; and combinations of these. In some embodiments, a first portion of ID 100 (e.g. a portion comprising one or more electrodes 111) is configured to be implanted in a first cardiac vein or other blood vessel (e.g. as listed hereabove) and/a second portion of ID 100 (e.g. a portion comprising one or more electrodes 111 and/or controller 130) is configured to be implanted in a second cardiac vein or other blood vessel (e.g. as listed hereabove). In some embodiments, a first portion of ID 100 (e.g. a portion comprising one or more electrodes 111) is configured to be implanted in a first cardiac vein (e.g. as listed hereabove), and a second portion of ID 100 (e.g. a portion comprising one or more electrodes 111) is configured to be implanted at an epicardial location, such as is described herebelow. In some embodiments, ID 100 is configured to be implanted in a blood vessel and/or other anatomical location such that electrodes 111 deliver stimulation energy to the left atrium, left ventricle, and/or other cardiac tissue (e.g. to deliver stimulation energy to one, two, or more locations of the left atrium or other cardiac locations). In some embodiments, ID 100 is configured to be implanted in one, two, or more locations selected from the group consisting of: middle and/or medial epicardial locations on the posterior wall of left atrium; superomedial epicardial locations on the roof of the left atrium; middle epicardial locations on the anterior wall of left atrium; medial (proximal) and/or middle and/or lateral (distal) locations within the coronary sinus (e.g. when affixed adjacent to the left atrial epicardium for left atrial pacing, and/or affixed adjacent to the left ventricular epicardium for left ventricular pacing); epicardial locations at the apex of the left ventricle and/or right ventricle; medial and/or middle epicardial locations on the anterior wall of the left ventricle and/or right ventricle; lateral epicardial locations on the left-lateral wall of the left ventricle and/or the right-lateral wall of the right ventricle; medial epicardial locations over the interventricular septum between the left and right ventricles; medial and/or middle epicardial locations on the posterior wall of the left ventricle and/or right ventricle; and combinations of these. One or more parameters of ID 100 and/or anchor 150 can be selected (e.g. in manufacturing and/or from a kit of two or more devices 100) based on one or more properties of the Vein of Marshall (e.g. the length and/or the diameter of the vein).

In some embodiments, electrodes 111 comprise a coating and/or a surface treatment (either or both, “coating” herein), such as a coating that is configured to enhance the recording ability of ID 100 via electrodes 111 and/or to enhance the pacing ability of ID 100. For example, electrode 111 can comprise one or more coatings that are configured to increase the surface area of electrodes 111, such as to enhance the recording ability of electrodes 111, such as to increase the charge-injection capacity for delivering a stimulation impulse, such as by lowering the resistive impedance and/or by increasing the capacitive impedance of electrodes 111.

ID 100 can comprise controller 130, which can be configured to perform various functions of ID 100. Controller 130 can comprise a microprocessor, memory, and other components that can be constructed and arranged to control, perform, and/or otherwise enable one or more functions of ID 100. In some embodiments, controller 130 comprises one or more algorithms, algorithm 135 shown. Controller 130 can be constructed and arranged to execute algorithm 135 and to thereby execute one or more functions of ID 100. In some embodiments, each electrode 111 of electrode array 110 is independently addressable (e.g. electrically connected to at least two wires, such as ground and power or data, between each electrode 111 and controller 130), such that signals (e.g. data and/or power) can be transmitted between controller 130 and each electrode 111 individually or collectively. Alternatively or additionally, controller 130 and/or electrode array 110 can be configured in a multiplexed arrangement, such that each electrode 111 can be individually addressed via a multiplexing component.

ID 100 can include transceiver 120. Transceiver 120 can be configured to communicate (e.g. wirelessly communicate) with one or more other components of system 10, for example, one or more additional implanted devices 100′, as well as EPD 200, CD 300, console 400, and/or or another component of system 10. Transceiver 120 can comprise a receiving and/or transmitting interface, antenna 125. Antenna 125 can comprise various shapes, for example, antenna 125 can comprise planar micro coils configured in various shapes.

ID 100 can include power module 140. Power module 140 can include one or more power-generating, power-harvesting, power-storing, and/or other power-supplying components configured to deliver energy to ID 100. Power module 140 can be configured to provide power to one or more components of ID 100. In some embodiments, power module 140 comprises one or more batteries, capacitors, and/or other power-storing devices. In some embodiments, ID 100 does not include a battery (i.e. a source of power that is generated by an electrochemical reaction), for example, when power module 140 is configured to harvest power (e.g. configured to harvest power transmitted wirelessly from EPD 200), and power module 140 is configured to directly provide the harvested power to power the various components of ID 100. Power module 140 can be constructed and arranged to “harvest” power from kinetic motion, for example, from kinetic motion of heart tissue when at least a portion of ID 100 is positioned on and/or within the heart. In some embodiments, power module 140 comprises one or more piezo electric components configured to convert kinetic energy to electrical energy.

In some embodiments, implantable device 100 can comprise patient sensor 160 shown. Patient sensor 160 can comprise one, two or more sensors selected from the group consisting of: accelerometer; position sensor; gravimetric sensor; pressure sensor; strain gauge; and combinations of these. System 10 can be configured to monitor one or more patient parameters based on information recorded by patient sensor 160, such as heartbeat, patient position, and/or patient activity.

ID 100 can include one or more functional elements, functional element 199 shown. Functional element 199 can comprise one, two, or more sensors selected from the group consisting of: pressure sensor such as blood pressure sensor; acoustic sensor; respiration sensor; gas sensor such as blood gas sensor; flow sensor such as blood flow sensor; temperature sensor; pH sensor; optical sensor; metabolic sensor, gravitational position sensor, gravitational orientation sensor, physical motion sensor; body position sensor; and combinations of these. In some embodiments, functional element 199 comprises one, two, or more transducers, such as an optical transducer (e.g. an LED). In some embodiments, functional element 199 comprises a fluid delivery assembly, such as an assembly configured to deliver a pharmaceutical drug or other agent to the patient. In these embodiments, functional assembly 199 can comprise a refill port, such as a port that can be accessed with a needle that is advanced through the patient's skin into functional assembly 199 in order to refill functional assembly 199 with additional agent to be delivered to the patient. In some embodiments, functional assembly 199 is configured to deliver energy to the patient, such as thermal energy, electrical energy, magnetic energy, radioactive energy, sound energy (e.g. ultrasound energy), light energy, mechanical energy, and/or chemical energy to the patient.

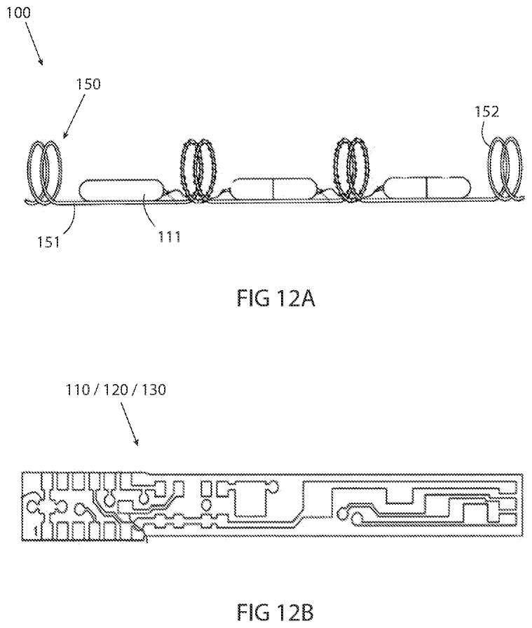

In some embodiments, ID 100 includes one or more circuit boards comprising one or more components of ID 100, for example electrode array 110, transceiver 120, controller 130, and/or other components of ID 100. In some embodiments, one or more circuit boards of ID 100 comprise flexible circuit boards, such as flexible printed circuit boards, for example as described in reference to FIG. 12 herein.

System 10 can be configured to both monitor one or more patient parameters and to treat the patient based on the monitored parameters. For example, system 10 can be configured to monitor (e.g. via electrode array 110) and analyze (e.g. via controller 130) electrograms recorded by ID 100, and to pace and/or otherwise stimulate tissue if atrial fibrillation (AF) is detected. In some embodiments, system 10 is configured to monitor and/or record one, two, or more of electrophysiological activity, patient temperature, heartbeat information, and/or another patient parameter.

Implantable device 100 can comprise one or more portions, such as a first portion implanted in a first location (e.g. a vein or other blood vessel), and a second portion implanted at another location (e.g. the epicardial surface of the heart or other anatomical location within the patient). Each portion can include one or more electrodes 111. In some embodiments, one portion (e.g. implanted in a coronary vein) comprises at least one, two or more electrodes 111, and another portion (e.g. positioned proximate but outside the heart) comprises at least controller 130.

External patient device 200 (EPD 200) can be constructed and arranged to be worn by the patient, such as when positioned on the skin of the patient (e.g. when EPD 200 is temporarily adhered or otherwise temporarily attached to the patient's skin), and/or when inserted in and/or otherwise attached to the patient's clothing. In some embodiments, EPD 200 includes attachment assembly 280. Attachment assembly 280 can include an adhesive, such as an adhesive patch, configured to adhere EPD 200 to the patient's skin for at least 6 hours, such as at least 12 hours, or at least 24 hours (e.g. before the adhesive patch must be replaced). Alternatively or additionally, attachment assembly 280 can comprise a harness, clip, specialized garment, or other non-adhesive based tool for positioning EPD 200 proximate the patient (e.g. proximate the location where ID 100 is implanted in the patient). For example, attachment assembly 280 can comprise a chest strap constructed and arranged to hold EPD 200 over the patient's heart, for example when ID 100 is implanted onto the epicardial surface of the patients left atrium.

EPD 200 can include transceiver 220. Transceiver 220 can be configured to communicate (e.g. wirelessly communicate) with one or more components of system 10, for example, one or more implanted devices 100, and/or one or more additional external patient devices 200′, as well as CD 300, console 400, and/or other components of system 10. Transceiver 220 can comprise a receiving and/or transmitting interface, antenna 225. EPD 200 can be constructed and arranged to transmit power and/or data to one or more implantable devices 100, such as by transmitting a radio frequency (RF) energy from antenna 225, through the skin of the patient, towards ID 100, and ID 100 can be constructed and arranged to harvest the RF energy and/or receive the RF data via antenna 125. In some embodiments, EPD 200 is constructed and arranged to receive data from one or more implantable devices 100, such as when transceiver 120 is constructed and arranged to transmit RF data to EPD 200.

EPD 200 can include one or more user interfaces, user interface 250 shown. User interface 250 can include one or more user input and/or user output components, for example, one or more: displays, indicators (e.g. LEDs), speakers, buttons, microphones, and/or other user interface components. In some embodiments, EPD 200 includes one or more functional elements, functional element 299 shown. Functional element 299 can include one or more sensors and/or transducers. In some embodiments, functional element 299 comprises a fluid delivery assembly, such as an assembly configured to deliver a pharmaceutical drug or other agent to the patient (e.g. through the skin of the patient). In these embodiments, functional assembly 299 can comprise a refill port, such as a port that can be accessed with a needle in order to refill functional assembly 299 with additional agent to be delivered to the patient. In some embodiments, functional assembly 299 is configured to deliver energy to the patient, such as thermal energy, electrical energy, magnetic energy, radioactive energy, sound energy (e.g. ultrasound energy), light energy, mechanical energy, and/or chemical energy to the patient.

EPD 200 can include processing unit 210 which can be configured to perform one or more functions of EPD 200. Processing unit 210 can include one or more algorithms, algorithm 215 shown. In some embodiments, processing unit 210 analyzes data (e.g. via algorithm 215) received from ID 100. For example, EPD 200 can receive data from ID 100, process (e.g. mathematically process) the information received via algorithm 215 (e.g. to determine if pacing should be performed, and to determine the parameters of stimulation energy to be delivered), and send information and/or power to ID 100 based on the processed information.

CD 300 can include one or more catheters and/or or one or more surgical tools for delivering ID 100 into the patient. Additionally, CD 300 can include one or more devices configured to diagnose and/or treat the patient, such as to perform a diagnosis and/or a treatment during a clinical procedure in which ID 100 is implanted into the patient. For example, CD 300 can comprise a cardiac mapping catheter which can be used to collect data (e.g. data to be processed by console 400) such as to map the cardiac electrical activity of the heart. Additionally or alternatively, CD 300 can comprise an ablation catheter which can be used to ablate tissue (e.g. cardiac tissue). In some embodiments, system 10 can include one or more clinician devices 300 that are constructed and arranged to enable the clinician to perform: a mapping procedure, a tissue treatment procedure (e.g. an ablation procedure or other tissue treatment procedure), a multi-site pacing procedure, and/or an ID 100 implantation procedure (e.g. for continued, post procedural treatment of the patient).

In some embodiments, CD 300 comprises electrode array 310 shown, which can comprise one or more arrays of electrodes that can be inserted into the patient. CD 300 can include user interface 350 shown. User interface 350 can include one or more user input and/or user output components, for example, one or more: displays, indicators (e.g. LEDs), speakers, buttons, levers, microphones, and/or other user interface devices. In some embodiments, user interface 350 comprises a handle (e.g. a catheter handle) including one or more controls, such as a steering control.

In some embodiments, CD 300 includes transceiver 320. Transceiver 320 can comprise an assembly configured to communicate (e.g. wirelessly communicate) with one or more components of system 10, for example, one or more implanted devices 100, one or more external patient devices 200, console 400, and/or other components of system 10. Transceiver 320 can comprise a receiving and/or transmitting interface, antenna 325. In some embodiments, CD 300 includes one or more functional elements, functional element 399 shown. Functional element 399 can include one or more sensors and/or transducers. In some embodiments, functional element 399 comprises a fluid delivery assembly, such as an assembly configured to deliver a pharmaceutical drug or other agent to the patient. In these embodiments, functional assembly 399 can comprise a refill port, such as a port that can be accessed with a needle in order to refill functional assembly 399 with additional agent to be delivered to the patient. In some embodiments, functional assembly 399 is configured to deliver energy to the patient, such as thermal energy, electrical energy, magnetic energy, radioactive energy, sound energy (e.g. ultrasound energy), light energy, mechanical energy, and/or chemical energy to the patient.

In some embodiments, system 10 includes a data storage and processing device, server 600. Server 600 can comprise an “off-site” server (e.g. outside of the operating room or other clinical site in which ID 100 is implanted), such as a server maintained by the manufacturer of system 10. Alternatively or additionally, server 600 can comprise a cloud-based server. Server 600 can include processing unit 610 shown, which can be configured to perform one or more functions of server 600. Processing unit 610 can include one or more algorithms, algorithm 615. Server 600 can be configured to receive and store various forms of data, such as: patient, procedural, device, and/or other information, data 620. Data 620 can comprise data collected from multiple patients (e.g. multiple patients treated with system 10), such as data collected during and/or after clinical procedures where device 100 was implanted into the patient. For example, data can be collected from ID 100, transmitted to EPD 200, and sent to server 600 for analysis. In some embodiments, one or more devices of system 10, such as EPD 200 and server 600, can communicate over a network, for example, a wide area network such as the Internet. In some embodiments, system 10 includes a virtual private network (VPN) through which various devices of system 10 transfer data.

Algorithm 615 can be configured to analyze data 620. For example, algorithm 615 can be configured to analyze data 620 collected from multiple patients to identify similarities and/or differences in treatment parameters and patient results. In some embodiments, algorithm 615 comprises a machine learning and/or other artificial intelligence algorithm (“AI algorithm” herein) that can be configured to identify patterns in the correlations between treatment parameters and results based on data collected from multiple patients. In some embodiments, algorithm 615 analyzes patterns to determine better treatment parameters for one or more patients to be treated using system 10. For example, algorithm 615 can identify one or more patterns in the data (e.g. one or more patterns associated with efficacy of the treatment being delivered to the patient) by analyzing data 620 collected from many patients (e.g. tens of thousands of patients). Algorithm 615 can be further configured to use these patterns to determine whether a patient (e.g. in the set of patients from which the data was collected and/or in a new patient) is receiving sub-optimal treatment (e.g. the parameters associated with pacing and/or other energy being delivered could be modified to improve efficacy). System 10 (e.g. via algorithm 615) can be configured to alert the clinician of a patient receiving sub-optimal treatment, and to recommend (e.g. via CD 300, such as the clinician's phone or computer) the parameters be adjusted. In some embodiments, the clinician may schedule an appointment to adjust the parameters (e.g. in person), or the parameters can be adjusted remotely, for example, when CD 300 is configured to adjust the parameters remotely via the network. Alternatively or additionally, server 600 can adjust the parameter automatically (e.g. via the network). In some embodiments, one or more parameters are automatically adjustable (e.g. within certain thresholds), while other parameters require clinician approval.

As described herein, system 10 can comprise one or more algorithms, such as algorithms 135, 215, 415, and 615 shown in FIG. 1. In some embodiments, one or more of these algorithms can comprise an AI algorithm. In some embodiments, one or more of these algorithms comprises a bias, such as a bias to tend toward classification of false positives and/or false negatives. In some embodiments, one or more of these algorithms comprises a bias toward false positive detection of an arrhythmia, such as to avoid not delivering stimulation energy when an arrhythmia is present (e.g. stimulate for all arrhythmia as well as some non-arrhythmia events that are classified as an arrhythmia event due to the false positive bias).

In some embodiments, device 100 comprises at least one sensing location (e.g. at least one electrode 111 is configured to sense or otherwise record the electrical activity of the heart). Additionally or alternatively, device 100 can comprise at least three pacing locations (e.g. at least two, or at least three electrodes 111 are configured to deliver energy to tissue to pace the heart). In some embodiments, controller 130 of device 100 includes a fibrillation and/or other arrhythmias detection algorithm (e.g. algorithm 135), that determines when a patient requires therapy (e.g. based on recorded electrical activity of the heart). Alternatively or additionally, algorithm 215 of processing unit of EPD 200 can comprise an arrhythmia detection algorithm.

In some embodiments, EPD 200 is configured to transmit a signal to ID 100 when pacing is required. For example, transceiver 220 can produce an electromagnetic and/or radiofrequency signal (e.g. a signal at a predetermined frequency) in response to the fibrillation/arrhythmia algorithm (e.g. algorithm 215) determining that pacing therapy is required. In some embodiments, ID 100 includes a stent-like structure (e.g. anchor 150) constructed and arranged to hold device 100 within a vessel (e.g. a cardiac vein) In some embodiments, device 100 is positioned within the vessel with at least one electrode 111 positioned on the vein wall, closest to the cardiac muscle.

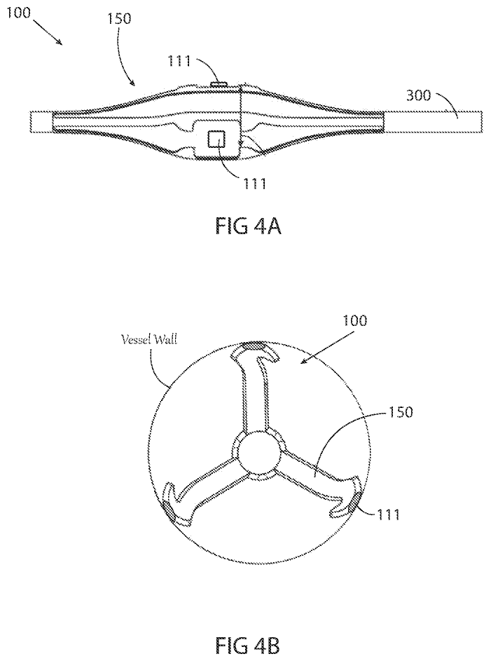

In some embodiments, anchor 150 comprises a stent-like construction. Device 100 can comprise an elongate shape, with a proximal end and a distal end (e.g. where the distal end is placed distally of the proximal end when ID 100 is implanted into a vessel). In some embodiments, the overall shape of ID 100 is defined by the shape of anchor 150, which can comprise an elongate shape including a proximal end and a distal end. In some embodiments, the distal end of ID 100 is configured to be placed inside a vein where the diameter of the vein is slightly smaller than the diameter of the vein where the proximal end of ID 100 is placed. In some embodiments, anchor 150 comprises a tapered design to accommodate this diameter delta, for example as shown in FIGS. 4A and 4B. Today's commercial stents have been used primarily to hold open an artery or vein, however in ID 100, anchor 150 is configured (e.g. solely configured) to anchor (e.g. chronically fixate) ID 100 inside the vessel in which it has been implanted.

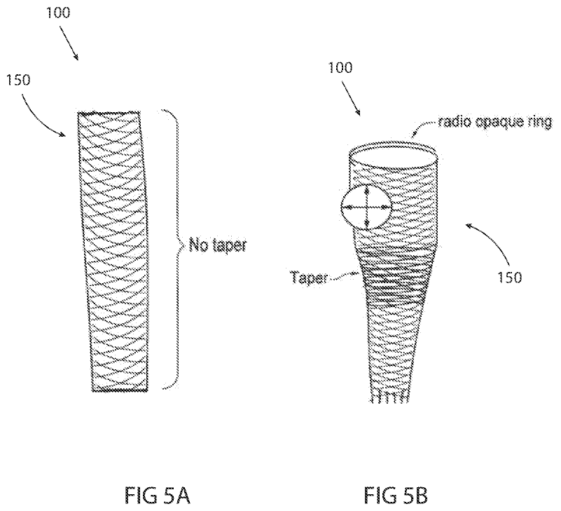

In some embodiments, anchor 150 comprises a stent assembly constructed and arranged to be easily inserted into a body lumen. In some embodiments, anchor 150 comprises an expandable construction (e.g. a self-expanding construction), such as a construction that expands when advanced from the lumen of a delivery catheter, such as is described in reference to FIGS. 5A, 5B, and/or 6 herein.

In some embodiments, anchor 150 comprises a stent assembly constructed and arranged to provide a constant pressure on the walls of the lumen in its expanded state to ensure ID 100 is maintained in its implanted position.

In some embodiments, anchor 150 comprises a stent assembly comprising a flexible body constructed and arranged to hold ID 100 (e.g. at least one electrode 111 of ID 100) against the lumen wall located on the cardiac muscle side while maintaining the compliance of the lumen.

In some embodiments, anchor 150 comprise a stent assembly having a smooth adherent surface, such as a surface configured to prevent fatty deposits and oils from sticking to ID 100.

In some embodiments, anchor 150 comprises a stent assembly constructed and arranged to retain the position of ID 100 within the lumen without causing damage to the lumen.

In some embodiments, anchor 150 comprises a stent assembly having an atraumatic structure (e.g. a structure that is flexible and soft enough, and free from sharp edges, such as to allow placement in a vein or other blood vessel, without damaging the blood vessel.

In some embodiments, anchor 150 comprises a stent assembly for placement in a body lumen, constructed and arranged to retain ID 100 within the body lumen in an open position. Anchor 150 can include a cylindrical shell and a plurality of circular coils embedded within the cylindrical shell. In some embodiments, anchor 150 is changeable between a first unexpanded state for placement within the body lumen and a second expanded state for holding ID 100 within the body lumen once positioned therein. In some embodiments, the cylindrical shell is made of expandable materials.

As described herein, system 10 can be constructed and arranged to deliver multi-site stimulation. System 10 can be configured to deliver stimulation energy of specific waveshapes, amplitudes, polarities, and durations and at specific times and through one or more specific channels, as determined by an algorithm of system 10, such as algorithm 135, 215, and/or 415, described herein. In some embodiments, system 10 delivers multi-site stimulation energy via ID 100 and/or CD 300. In some embodiments, system 10 performs an assessment of the patient's tissue (e.g. the tissue proximate the multiple stimulation sites) and determines a set of patient-specific optimized pacing parameters based on the assessment (e.g. as performed by an algorithm of system 10). These parameters can be determined during a clinical procedure, and these parameters can be programmed into EPD 200 and/or ID 100 for configuring a future stimulation to be delivered (e.g. when EPD 200 and/or ID 100 are used chronically following the clinical procedure).

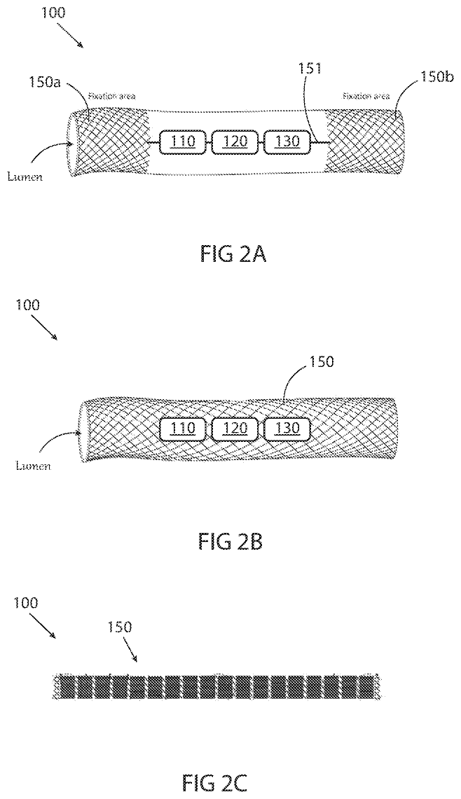

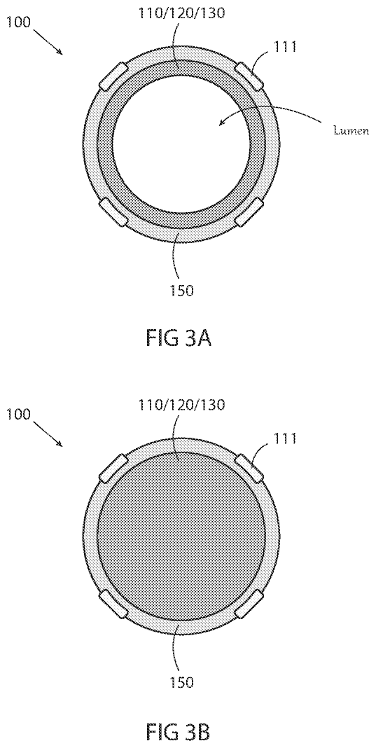

Referring now to FIG. 2A, a side view of an implantable device including two anchoring assemblies is illustrated, consistent with the present inventive concepts. Implantable device 100 of FIG. 2A can be of similar construction and arrangement, and can comprise similar components, to ID 100 described in reference to FIG. 1 herein. In some embodiments, ID 100 is shown in a deployed (expanded) state and includes an anchor 150 comprising a first anchor 150a and a second anchor 150b positioned on each end of ID 100, as shown in FIG. 2A. Anchors 150a and 150b can be connected via a filament or other linkage, connector 151. In some embodiments, anchors 150a and 150b can each comprise a stent-like structure, such as a balloon-expandable and/or self-expanding stent structure comprising a lumen therethrough. In some embodiments, anchors 150a and/or 150b comprise a wire mesh construction. One or more components of ID 100, such as electrode array 110, transceiver 120, and/or controller 130, can be fixedly attached to connector 151, and can be positioned between anchors 150a and 150b. In some embodiments, components 110, 120, and 130, as included in ID 100 of FIG. 2A, comprise a collective geometry such that there is a lumen through the complete length of ID 100, as shown in FIG. 3A, such that blood or other fluid can flow through device ID 100. Alternatively, components 110, 120, and/or 130, as included in ID 100 of FIG. 2A, comprise a collective geometry such that there is no lumen through ID 100, as shown in FIG. 3B, such that ID 100 blocks the flow of fluid in the vein or other anatomical location in which it is implanted. In either of these embodiments, electrode array 110 can comprise one or more electrodes 111, that are positioned on the outer diameter of anchor 150 as shown in FIGS. 3A-B, such that electrodes 111 contact tissue (e.g. contact the wall of the blood vessel into which ID 100 is implanted).

Referring now to FIG. 2B, a side view of an implantable device including a stent-like anchoring assembly is illustrated, consistent with the present inventive concepts. Implantable device 100 of FIG. 2B can be of similar construction and arrangement, and can comprise similar components, to ID 100 described in reference to FIG. 1 herein. ID 100 of FIG. 2B is shown in a deployed (expanded) state and includes an anchor 150 comprising an elongate stent-like structure, such as an expandable stent comprising a lumen therethrough and extending from the proximal to the distal end of ID 100 as shown. Anchor 150 can be constructed and arranged to contract in length when transitioning to an expanded diameter (e.g. when transitioning from a compacted diameter state, such as is shown in FIG. 2C, to a deployed, expanded diameter state such as is shown in FIG. 2B). In these embodiments, anchor 150 can be constructed and arranged to contract at least 50%, for example from 12 inches to 3 inches. One or more components of ID 100, such as electrode array 110, transceiver 120, and/or controller 130, can be fixedly attached to anchor 150. In some embodiments, anchor 150 comprises a wire mesh construction. In some embodiments, components 110, 120, and 130, as included in ID 100 of FIG. 2B, comprise a collective geometry such that there is a lumen through the complete length of ID 100, as shown in FIG. 3A, such that blood or other fluid can flow through device ID 100. Alternatively, components 110, 120, and/or 130, as included in ID 100 of FIG. 2B, comprise a collective geometry such that there is no lumen through ID 100, as shown in FIG. 3B, such that ID 100 blocks the flow of fluid in the vein or other anatomical location in which it is implanted. In either of these embodiments, electrode array 110 can comprise one or more electrodes 111, that are positioned on the outer diameter of anchor 150 as shown in FIGS. 3A-B, such that electrodes 111 contact tissue (e.g. contact the wall of the blood vessel into which ID 100 is implanted).

Referring now to FIG. 2C, a side view of the implantable device of FIG. 2B in a radially collapsed configuration is illustrated, consistent with the present inventive concepts. ID 100 includes a single anchoring element, anchor 150 shown, which can comprise a stent-like structure, such as a balloon-expandable and/or self-expanding stent structure. Other components of ID 100, such as are described herein, have been removed from FIG. 2C for illustrative clarity. ID 100 can be configured in the collapsed configuration shown in FIG. 2C for insertion into the patient (e.g. via the patient's vasculature system), after which it can be advanced to its deployment location and expanded, as described herein.

Referring now to FIGS. 4A and 4B, side and sectional views, respectively, of an implantable device comprising a basket-like anchoring element are illustrated, consistent with the present inventive concepts. Implantable device 100 of FIGS. 4A and 4B can be of similar construction and arrangement, and can comprise similar components, to ID 100 described in reference to FIG. 1 herein. Other components of ID 100, such as are described herein, have been removed from FIGS. 4A-B for illustrative clarity. FIG. 4A shows an ID 100 extending from the distal end of a clinician device 300 (e.g. a delivery catheter). Anchor 150 of ID 100 can comprise a basket-like construction, such as a basket comprising multiple expandable splines (e.g. 3 splines shown). In some embodiments, the splines of anchor 150 are resiliently biased in an expanded state, such anchor 150 expands to exert a force on the walls of the vessel into which ID 100 is implanted, as shown in FIG. 4B. In some embodiments, the splines of anchor 150 are plastically deformable, such as to allow expansion via a balloon or other radially deployable assembly. In some embodiments, anchor 150 comprises a wire-form basket. In some embodiments, anchor 150 is configured to transition from a compacted state to an expanded state without more than a 50% change in length. In some embodiments, electrodes 111 are positioned on the outer surface of the splines of anchor 150, such that electrodes 111 are maintained in contact with the vessel wall when ID 100 is deployed and implanted in the patient. Anchor 150 can be released from (e.g. disconnected from or pushed out of a lumen of) clinician device 300 such that CD 300 can be removed once ID 100 has been released.

Referring now to FIGS. 5A and 5B, side views of two implantable devices, each including a single anchoring element, are illustrated, consistent with the present inventive concepts. FIG. 5A shows anchor 150 comprising a stent-like construction. ID 100 of FIGS. 5A-B each include an anchor element 150 as shown, while other components of ID 100, as described herein, have been removed for illustrative clarity. Anchor 150 of FIG. 5A comprises uniform diameter along its length. FIG. 5B shows an anchor 150 also comprising a stent-like construction, but with a tapered diameter along its length (e.g. a taper that accommodates implantation of ID 100 into a vessel comprising a tapered diameter). In some embodiments, at least a portion of ID 100 can comprise a radiopaque portion, for example a radiopaque ring portion on an end (as shown) or other location of anchor 150.

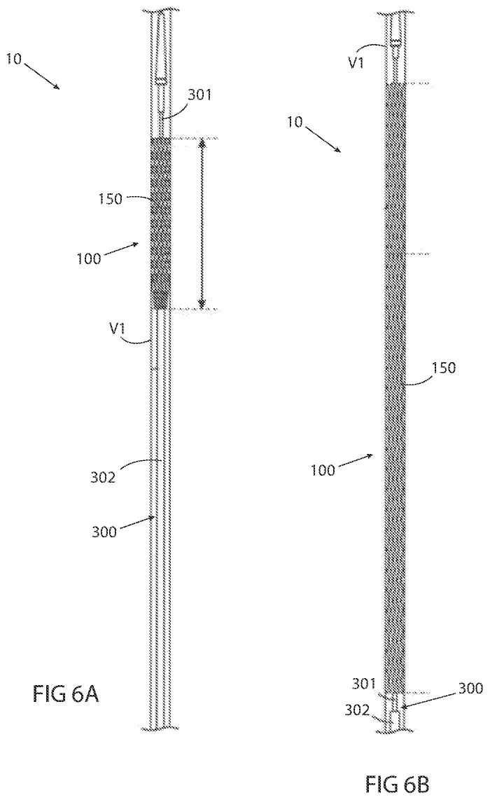

Referring now to FIGS. 6A and 6B, anatomical side views of a partially deployed and a fully deployed implantable device are illustrated, respectively, consistent with the present inventive concept. System 10 of FIGS. 6A and 6B can be of similar construction and arrangement, and can comprise similar components, to system 10 described in reference to FIG. 1 herein. ID 100 of FIGS. 6A and 6B includes anchor 150 as shown, and other components (e.g. electrode array 110, transceiver 120, and/or controller 130) which have been removed for illustrative clarity. In FIG. 6A, ID 100 has been partially deployed from CD 300. CD 300 includes inner shaft 301 and outer shaft 302, where ID 100 is captured between shafts 301 and 302 prior to deployment. In FIG. 6A, outer shaft 302 has been partially retracted, deploying a distal portion of ID 100. In FIG. 6B, outer shaft 302 has been fully retracted, causing the entire length of ID 100 to be deployed (e.g. expanded to contact the walls of blood vessel V1).

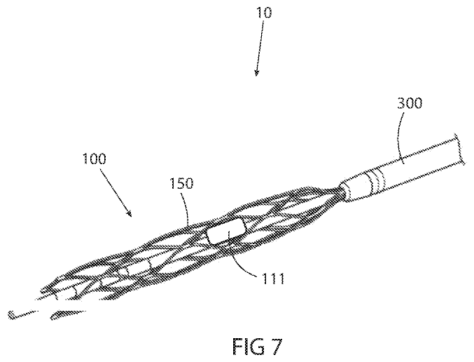

Referring now to FIG. 7, a perspective view of an implantable device being deployed from the distal portion of a clinician device is illustrated, consistent with the present inventive concepts. System 10 of FIG. 7 can be of similar construction and arrangement, and can comprise similar components, to system 10 described in reference to FIG. 1 herein. Anchor 150 of ID 100 of FIG. 7 can comprise an expandable braid-like construction as shown, for example a resiliently biased braid construction and/or a balloon-expandable construction. Other components of ID 100, such as are described herein, have been removed from FIG. 7 for illustrative clarity. In FIG. 7, implantable device 100 is shown partially deployed from clinician device 300. In some embodiments, one or more electrodes 111 or other components of ID 100 are positioned in the open cells of the braid as shown, such as one or more electrodes 111 configured to contact the wall of a vein or other blood vessel of the patient when ID 100 is implanted in the patient.

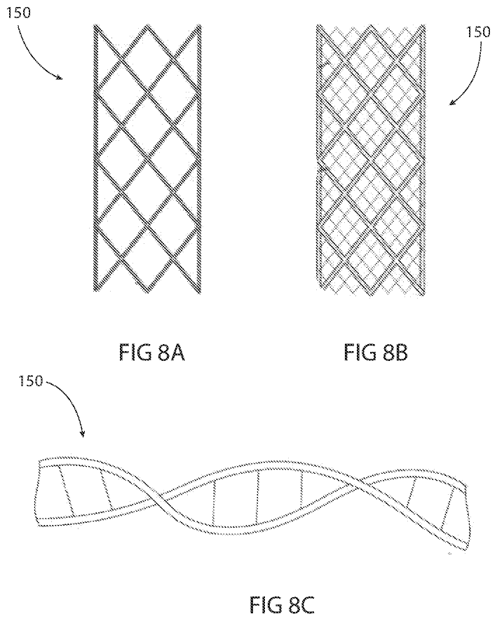

Referring now to FIGS. 8A, 8B, and 8C, side views of various embodiments of stent-like anchoring elements for an implantable device are illustrated, consistent with the present inventive concepts. One, two or more anchoring elements 150 of FIGS. 8A-C can be included in an ID 100, such as an ID 100 including electrode array 110, transceiver 120, and/or controller 130, as described herein. Anchoring element 150 of FIG. 8A comprises the braid construction shown, and it includes wires of a single diameter. Anchoring element 150 of FIG. 8B comprises the braid construction shown, and it includes wires of multiple diameters. Anchoring element of FIG. 8C comprises the helical coil construction shown.

Referring now to FIG. 9, a perspective view of an anchoring element for an implantable device is illustrated, consistent with the present inventive concepts. In some embodiments, anchor 150 of FIG. 9 comprises a flared end as shown, such that anchor 150 can be secured proximate the ostium of a vessel (e.g. where the flared portion of anchor 150 is deployed in the ostium). Anchor 150 of FIG. 9 can be included in an ID 100, such as an ID 100 including electrode array 110, transceiver 120, and/or controller 130, as described herein.

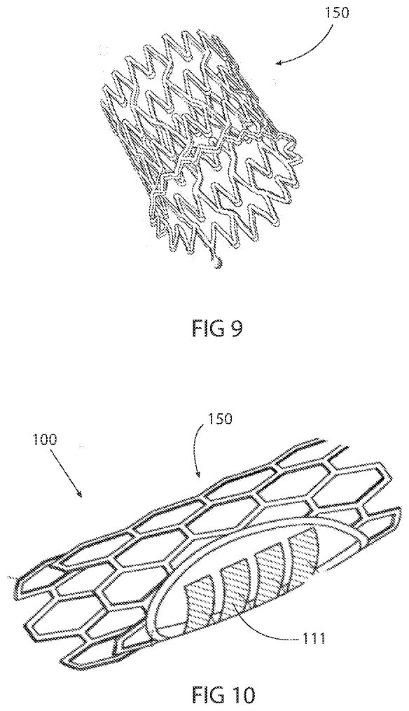

Referring now to FIG. 10, a perspective view of an implantable device is illustrated, consistent with the present inventive concepts. Implantable device 100 of FIG. 10 can be of similar construction and arrangement, and can comprise similar components, to ID 100 described in reference to FIG. 1 herein. Implantable device 100 can comprise one or more electrodes 111 (e.g. of an electrode array 110) positioned on the outer surface of anchor 150, as shown. Implantable device 100 can comprise other components, not shown for illustrative clarity, such as transceiver 120 and/or controller 130.

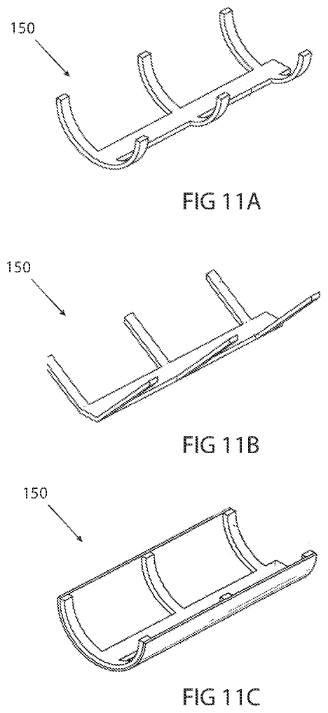

Referring now to FIGS. 11A, 11B, and 11C, perspective views of three non-circumferential anchoring elements are illustrated, consistent with the present inventive concepts. In some embodiments, anchor 150 comprises a partial circumferential (e.g. less the) 360° profile, for example as shown in FIGS. 11A-C. In some embodiments, one or more electrodes 111 and/or other component of ID 100 is positioned on a surface of anchor 150 (e.g. positioned on a surface of anchor 150 that is configured to make contact with a blood vessel wall or other solid tissue of the patient when ID 100 is implanted in the patient).

Referring now to FIG. 12A, a side view of an implantable device is illustrated, consistent with the present inventive concepts. Implantable device 100 of FIG. 12A can be of similar construction and arrangement, and can comprise similar components, to ID 100 described in reference to FIG. 1 herein. Anchor 150 of ID 100 can comprise one or more loops, loops 152, such as one or more sets of one or more loops, such as the four sets of two loops 152 shown in FIG. 12A. Loops 152 can be interconnected by one or more elongate connecting elements, connectors 151. In some embodiments, anchor 150 comprises an elongate filament (e.g. a single filament comprising a shape memory alloy) that has been resiliently biased (e.g. heat formed) into the desired geometry of anchor 150. For example, one, two, or more filaments (e.g. a single filament) comprising a nickel titanium alloy can be shaped into the loops 152 and connectors 151 shown in FIG. 12A. One or more components of ID 100 can be fixedly attached to connectors 151 and/or loops 152 of anchor 150, for example electrodes 111 shown. In some embodiments, various components of ID 100 (e.g. transceiver 120 and/or controller 130) are interconnected via one or more wires. In these embodiments, one or more wires can be positioned in a spiral arrangement around loops 152 as shown.

Referring now to FIG. 12B, a top view of a circuit board of an implantable device is illustrated, consistent with the present inventive concepts. Implantable device 100 of FIG. 12B can be of similar construction and arrangement, and can comprise similar components, to ID 100 described in reference to FIG. 1 herein. In some embodiments, ID 100 includes one or more circuit boards comprising one or more components of ID 100, for example electrode array 110, transceiver 120, controller 130, and/or other components of ID 100. In some embodiments, one or more circuit boards of ID 100 comprise flexible circuit boards, such as flexible printed circuit boards.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A system for providing therapy to a heart of a patient, the system comprising:
an implantable device configured to be implanted proximate the heart of the patient, the implantable device comprising:
an anchoring element configured to maintain the position of the implantable device after implantation in the patient;
at least one sensing electrode configured to sense the electrical activity of the heart;
at least three pacing electrodes configured to deliver electrical energy to the tissue of the heart; and
a controller including an algorithm, wherein the algorithm is configured to determine when the patient requires therapy; and
an external device comprising a transceiver configured to transmit energy to the implantable device,
wherein the implantable device does not comprise a battery.

2-12. (canceled)
